(19) United States
(12) Patent Application Publication
(10) Pub. No.: US 2018/0035524 A1
(43) Pub. Date: Feb. 1, 2018

(54) RADIATION IRRADIATION DEVICE

(71) Applicant: FUJIFILM Corporation, Tokyo (JP)

(72) Inventor: Masayoshi MATSUURA, Kanagawa (JP)

(73) Assignee: FUJIFILM Corporation, Tokyo (JP)

(21) Appl. No.: 15/628,598

(22) Filed: Jun. 20, 2017

(30) Foreign Application Priority Data
Jul. 26, 2016 (JP) 2016-145895

(51) Int. Cl. H05G 1/54; A61B 6/00; H05G 1/26; H05G 1/32

(52) U.S. Cl. CPC H05G 1/54; H05G 1/32; A61B 6/56; H05G 1/265

(57) ABSTRACT

Provided is a radiation irradiation device that prevents degradation of a battery resulting from a high current that flows when radiation is generated. The radiation irradiation device includes a radiation generating part that generates radiation; a battery part that supplies electric power to the radiation generating part; and an exposure switch that receives an emission instruction of the radiation from the radiation generating part. The battery part has a lithium ion battery, a capacitor connected in parallel to the lithium ion battery, a switch element that performs switching from a state where electric power is supplied from the lithium ion battery to the capacitor to a state where electric power is supplied from the capacitor to the radiation generating part. The switch element switches between the states of the electric power supply according to an instruction received in the exposure switch.

### CROSS-REFERENCE TO RELATED APPLICATIONS

The present application claims priority under 35 U.S.C. §119 to Japanese Patent Application No. 2016-145895, filed on Jul. 26, 2016. Each of the above application(s) is hereby expressly incorporated by reference, in its entirety, into the present application.

### BACKGROUND OF THE INVENTION

### 1. Field of the Invention

The present invention relates to a radiation irradiation device having a radiation source that receives electric power supply from a battery.

### 2. Description of the Related Art

In the related art, portable radiation irradiation devices used in a case where a patient's radiation image are captured in operating rooms, examination rooms, or inpatient rooms have been suggested variously.

The portable radiation irradiation devices basically include a leg part enabled to travel by wheels, a main body part that houses a control part consisting of a battery for driving a radiation source, an electric circuit related to the driving of the radiation source, and the like and is held on the leg part, and an arm part connected to the main body part, and are configured by attaching the radiation source to a tip of the arm part.

When such radiation irradiation devices are used, a radiation irradiation device is first moved to the vicinity of a patient's bed. Next, the radiation source is moved to a desired position, and a radiation detector is moved to a desired position behind a subject. Then, in this state, the subject is irradiated with radiation by driving the radiation source, and a radiation image of the subject is acquired by detecting the radiation transmitted through the subject using the radiation detector.

Here, in the related art, in the portable radiation irradiation devices, lead storage batteries are used as batteries. However, in a case where the lead storage batteries are frequently charged, degradation of the batteries becomes early due to a memory effect, and energy density is small. Therefore, there are problems in that the weight becomes heavy.

Thus, it is suggested that lithium ion batteries are used as the batteries of the radiation irradiation devices (for example, refer to JP2013-180059A, JP2010-273827A, and JP2014-150948A).

### SUMMARY OF THE INVENTION

However, even in a case where the lithium ion batteries are used, there are several problems. The lithium ion batteries have large internal resistance because the lithium ion batteries are connected in series. Hence, in a case where a high current is sent through a radiation source when generating radiation, a voltage drop of the lithium ion batteries becomes large, and becomes equal to or lower than a lower limit of battery rating. As a result, the lifespan of the lithium ion batteries becomes short.

Additionally, if the number of lithium ion batteries is increased by connecting the lithium ion batteries more in series, the value of a current of each lithium ion battery can be held down. However, due to the serialization, internal resistance becomes large, and the voltage drop increases.

The invention is to provide a radiation irradiation device that can prevent degradation of a battery resulting from a high current that flows when radiation is generated, in view of the above problems.

A radiation irradiation device of the invention includes a radiation generating part that generates radiation; a battery part that supplies electric power to the radiation generating part; and an emission instruction receiving part that receives an emission instruction of the radiation from the radiation generating part. The battery part has a storage battery, a capacitor connected in parallel to the storage battery, a switching part that performs switching from a state where electric power is supplied from the storage battery to the capacitor to a state where electric power is supplied from the capacitor to the radiation generating part. The switching part switches between the states of the electric power supply according to an instruction received at the emission instruction receiving part.

Additionally, in the radiation irradiation device of the above invention, the switching part can have a switch element connected between the storage battery and the capacitor.

Additionally, in the radiation irradiation device of the above invention, the emission instruction receiving part can receive two-step instructions of an emission preparation instruction of the radiation and an emission instruction of the radiation, and the switching part can be brought into a state where electric power is supplied from the storage battery to the capacitor, according to the emission preparation instruction of the radiation.

Additionally, the radiation irradiation device of the above invention can further include a notification part that performs notification of charging from the storage battery to the capacitor being completed.

Additionally, in the radiation irradiation device of the above invention, the notification part can include a light-emitting part that emits light when the charging from the storage battery to the capacitor is completed.

Additionally, the radiation irradiation device of the above invention can further include a reverse current suppressing part that suppresses a reverse current from the capacitor to the storage battery.

Additionally, in the radiation irradiation device of the above invention, the reverse current suppressing part can have a diode element.

Additionally, the radiation irradiation device of the above invention can further include an inrush current suppressing part that suppresses an inrush current from the storage battery to the capacitor.

Additionally, in the radiation irradiation device of the above invention, the inrush current suppressing part can have a resistance element.

Additionally, in the radiation irradiation device of the above invention, an electric double layer capacitor can be used as the capacitor.

Additionally, in the radiation irradiation device of the above invention, a lithium ion battery can be used as the storage battery.

According to the radiation irradiation device of the invention, the battery part has the storage battery, the capacitor connected in parallel to the storage battery, the switching part that performs the switching from the state where electric power is supplied from the storage battery to the capacitor to the state where electric power is supplied from the capacitor to the radiation generating part. The switching part switches between the states of the electric power supply according to the instruction received at the emission instruction receiving part. Hence, since electric power is not directly supplied from the storage battery to the radiation generating part but electric power is supplied to the radiation generating part with the discharge voltage of the capacitor when radiation is generated from the radiation generating part, degradation resulting from a voltage drop of the storage battery can be prevented.

Additionally, since the states of the electric power supply are switched therebetween according to the instruction received at the emission instruction receiving part, the discharge voltage of the capacitor can be supplied to the radiation generating part at a user's desired timing.

### BRIEF DESCRIPTION OF THE DRAWINGS

FIG. 1 is a perspective view illustrating an entire shape of a radiation irradiation device of an embodiment of the invention.

FIG. 2 is a view illustrating the state when the radiation irradiation device of the embodiment of the invention is used.

FIG. 3 is a view of a leg part as seen from below.

FIG. 4 is a schematic view illustrating an electrical configuration of a battery part and a radiation generating part.

FIG. 5 is a timing chart for explaining the operation from charging to a capacitor of the battery part to exposure of radiation.

FIG. 6 is a view illustrating another embodiment of the battery part.

FIG. 7 is a view illustrating an example of a gate voltage applied to a switch element.

FIG. 8 is a view illustrating still another embodiment of the battery part.

FIG. 9 is a view of the radiation irradiation device illustrated in FIG. 1 as seen from the front.

FIG. 10 is an external perspective view of a radiation detector as seen from a radiation detection surface side.

### DESCRIPTION OF THE PREFERRED EMBODIMENTS

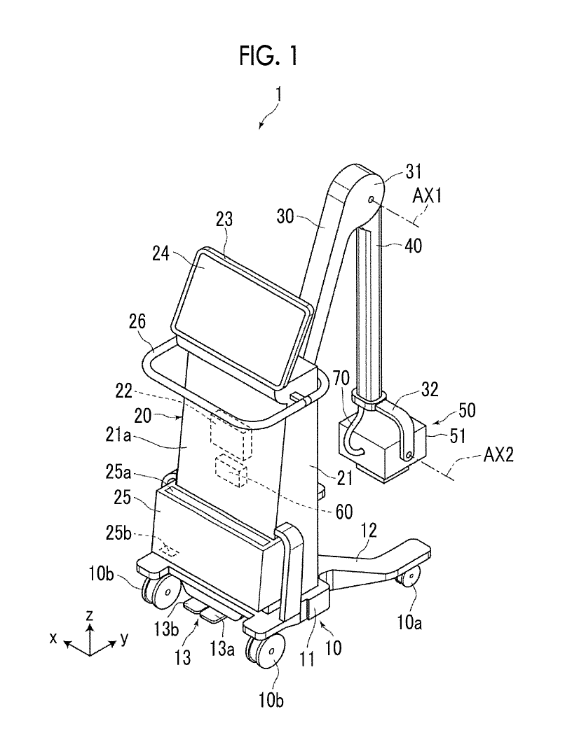

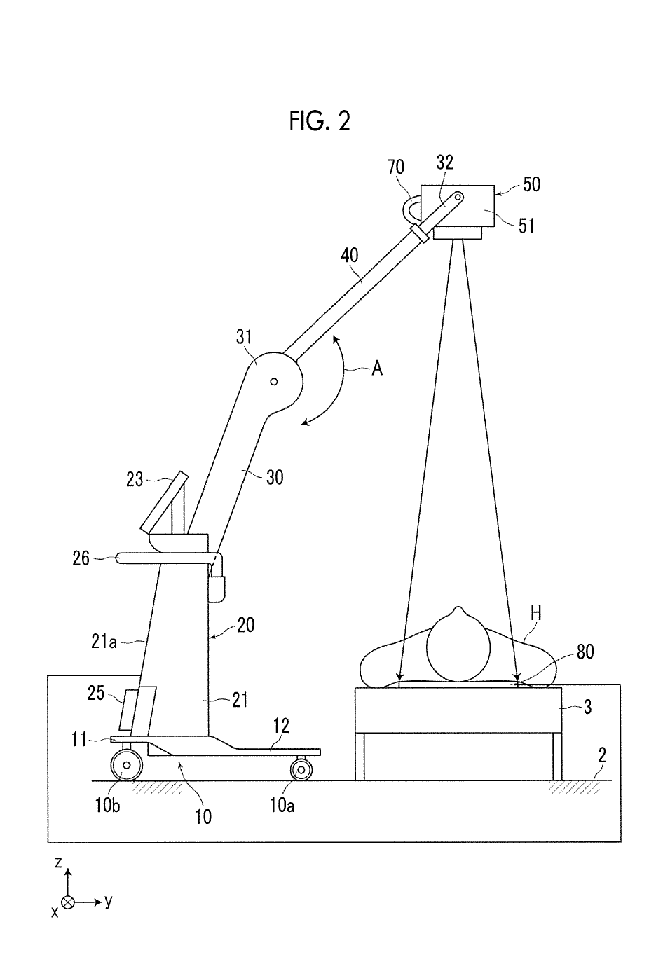

Hereinafter, a radiation irradiation device of an embodiment of the invention will be described in detail, referring to the drawings. Although the invention has features in the configuration of electric power supply to the radiation generating part in the radiation irradiation device, the entire configuration of the radiation irradiation device will first be described. FIG. 1 is a perspective view illustrating the entire shape of the radiation irradiation device of the present embodiment when being not used, and FIG. 2 is a side view illustrating the state when the radiation irradiation device of the present embodiment is used. In addition, in the following, an upper side and a lower side in the vertical direction in a state where the radiation irradiation device is placed on, for example, a device placement surface, such as a floor of a medical institution, are referred to as “up” and “down”, respectively, and a direction perpendicular to the vertical direction in the same state is referred to as a “horizontal” direction. Additionally, in the views to be described below, the vertical direction is defined as a z direction, a leftward-rightward direction of the radiation irradiation device is defined as an x direction, and a forward-backward direction of the radiation irradiation device is defined as a y direction. In addition, the front herein means a side to which an arm part extends from a main body part of the radiation irradiation device when the device is used.

As illustrated in FIGS. 1 and 2, a radiation irradiation device 1 of the present embodiment includes a leg part 10, a main body part 20, a supporting member 30, an arm part 40, and a radiation generating part 50.

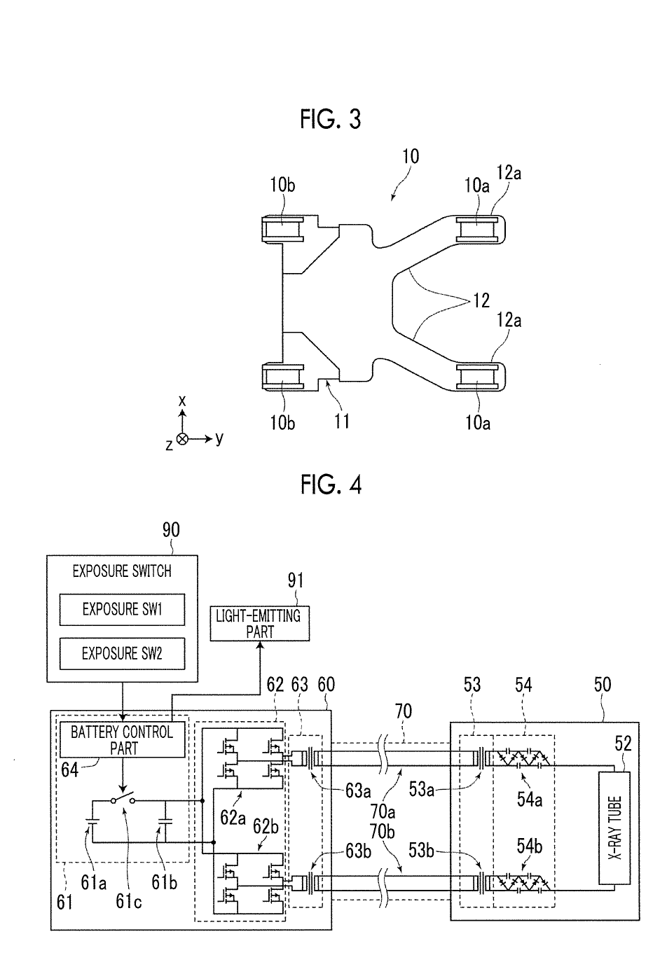

The leg part 10 is capable of traveling on a device placement surface 2, and consists of a plate-shaped pedestal part 11 on which the main body part 20 is placed, and a foot arm part 12 that extends from the pedestal part 11 toward the front. FIG. 3 is a view of the leg part 10 as seen from below. As illustrated in FIG. 3, the foot arm part 12 is formed in a V shape that widens in the leftward-rightward direction toward the front. First casters 10a are respectively provided on bottom surfaces of two tip parts 12a at the front of the foot arm part 12, and second casters 10b are respectively provided on bottom surfaces of two corners at the rear of the pedestal part 11. By forming the foot arm part 12 in a V shape as described above, for example, as compared to a case where the entire leg part 10 is formed in a rectangular shape, an edge part of the leg part does not easily collide against its surrounding obstacle when the leg part 10 is rotated. Thus, handling can be made easy. Additionally, weight reduction can also be achieved.

Each first caster 10a has a shaft that extends in the upward-downward direction, and is attached to the foot arm part 12 such that a rotating shaft of a wheel is revolvable within a horizontal plane about the shaft of the first caster. Additionally, each second caster 10b also has a shaft that extends in the upward-downward direction, and is attached to the pedestal part 11 such that a rotating shaft of a wheel is revolvable within the horizontal plane about the shaft of the second caster. In addition, the rotating shaft of each wheel herein is a rotating shaft when the wheel rotates and travels. The leg part 10 is configured so as to be capable of traveling in an arbitrary direction on the device placement surface 2 by the first casters 10a and the second casters 10b.

Additionally, as illustrated in FIG. 1, a pedal part 13 is provided at the rear of the leg part 10. The pedal part 13 consists of two pedals of a first pedal 13a and a second pedal 13b. The first pedal 13a is a pedal for bringing the second casters 10b into a non-revolvable state. As a user steps on the first pedal 13a, the second casters 10b are configured so as to be locked in revolution by a locking mechanism and brought into the non-revolvable state.

Additionally, the second pedal 13b is a pedal for bringing the second casters 10b into a revolvable state from the non-revolvable state. As the user steps on the second pedal 13b, the second casters 10b are configured so as to be released from the locking by the locking mechanism and brought into the revolvable state again.

A well-known configuration can be used as the locking mechanism that locks the revolution of the second casters 10b. For example, the revolution may be locked such that both sides of the wheels of the second casters 10b are sandwiched by plate-shaped members, or the revolution may be locked by providing members that stop the rotation of shafts of the second caster 10b that extend in the upward-downward direction.

The main body part 20 is placed on the pedestal part 11 of the leg part 10, and includes a housing 21. A control part 22 that controls driving of the radiation irradiation device 1 and an electric power supply part 60 are housed within the housing 21.

The control part 22 performs control regarding generation and irradiation of radiation, such as a tube current, irradiation time, and a tube voltage, in the radiation generating part 50, and control regarding acquisition of radiation images, such as image processing of a radiation image acquired by the radiation detector to be described below. The control part 22 is configured of, for example, a computer in which a program for control is installed, exclusive hardware, or combination of both.

The electric power supply part 60 supplies electric power to the radiation generating part 50, a monitor 23, and the radiation detector housed within, a cradle 25 to be described below. In addition, the monitor 23 may be configured so as to be attachable to and detachable from the main body part 20. In that case, the electric power supply part 60 supplies electric power to a battery built in the monitor 23 to charge the battery. Additionally, the radiation detector also has a battery built therein, and the electric power supply part 60 supplies electric power to the built-in battery to charge the battery.

FIG. 4 is a schematic view illustrating an electrical configuration of the electric power supply part 60 and the radiation generating part 50. As illustrated in FIG. 4, the electric power supply part 60 includes a battery part 61, an inverter circuit part 62, and a first booster circuit part 63.

The battery part 61 includes a lithium ion battery 61a, a capacitor 61b, a switch element 61c, and a battery control part 64.

The lithium ion battery 61a is equivalent to a storage battery of the invention and is a cell obtained by connecting a plurality of lithium ion batteries in parallel. The lithium ion battery 61a of the present embodiment outputs a voltage of 48 V. Although the voltage output from the lithium ion battery 61a is not limited to 48 V, it is desirable that this voltage is 60 V or less. By setting the voltage to 60 V or less, the insulation creepage space distance can be made small, and size reduction can be achieved.

Additionally, although one lithium ion battery is used in the present embodiment, the invention is not limited to this. Two or more lithium ion batteries may be connected in parallel and used. In this case, in the plurality of lithium ion batteries, it is preferable to short-circuit the same poles. Noise can be reduced by connecting the lithium ion batteries in this way.

Additionally, by connecting the lithium ion batteries in parallel in this way, as compared to a case where lithium ion batteries are connected in series, the insulation creepage space distance can be made small, and size reduction can be achieved. However, two or more lithium ion batteries may be connected in series.

Additionally, in the present embodiment, the lithium ion battery is used as the storage battery from a viewpoint of weight reduction and easy handling. However, the invention is not limited to this. A battery consisting of a nickel hydride battery, a battery consisting of a NaS battery, a battery consisting of a fuel cell, and the like can be used. In addition, the storage battery may not be necessarily installed within a main body part 20. For example, storage batteries of electric automobiles may be used.

The capacitor 61b is connected in parallel to the lithium ion battery 61a, and is charged by the lithium ion battery 61a. Although it is preferable to use an electric double layer capacitor as the capacitor 61b, the invention is not limited to this, and an electrolytic capacitor may be used. As the capacity of the capacitor 61b, it is desirable to set the capacity such that the voltage output from the electric power supply part 60 becomes 4 times or more and 6 times or less the output voltage of the lithium ion battery 61a.

By setting the voltage output from the electric power supply part 60 to 4 times or more the output voltage of the lithium ion battery 61a, a resistance against the noise from the outside when going via the cable part 70 to be described below can be made stronger. Additionally, by setting the voltage output from the electric power supply part 60 to 6 times or less the output voltage of the lithium ion battery 61a, it is not necessary to use a high-voltage cable as the cable part 70, and reduction of cost can be achieved. Moreover, since wiring line coating of the cable part 70 can be made thin, the degree of freedom of the cable part 70 can be improved. Accordingly, the movement of the arm part 40 (to be described below) in which the cable part 70 extends is can be made smooth. Specifically, it is desirable that the voltage output from the electric power supply part 60 is 60 V or more and 300 V or less. In the present embodiment, the voltage output from the electric power supply part 60 is set to 250 V.

The switch element 61c is connected between the lithium ion battery 61a and the capacitor 61b, and is turned on and off according to the operation of an exposure switch 90 to be described below. As the switch element 61c, for example, it is preferable to use a semiconductor switch, such as an FET (field effect transistor) switch. However, the invention is not limited to this, and a mechanical switch, such as a relay, may be used.

The capacitor 61b is charged by the lithium ion battery 61a while the switch element 61c is turned on and when the switch element 61c is turned off, the voltage charged by the capacitor 61b is discharged.

The battery control part 64 controls ON and OFF states of the switch element 61c according to the operation of the exposure switch 90. Specifically, in the present embodiment, an FET switch is used as the switch element 61c, and the battery control part 64 applies a gate voltage to a gate of the FET switch according to the operation of the exposure switch 90. In addition, in the present embodiment, the switch element 61c and the battery control part 64 are equivalent to a switching part of the invention.

The inverter circuit part 62 converts a direct current voltage discharged from the capacitor 61b of the battery part 61 into an alternating voltage. Specifically, the inverter circuit part 62 includes a positive electrode side inverter circuit 62a and a negative electrode side inverter circuit 62b. In addition, the circuit configuration of the inverter circuits is not limited to the circuit configuration illustrated in FIG. 4, and other well-known inverter circuits may be adopted.

The first booster circuit part 63 boosts an alternating voltage output from the inverter circuit part 62. Specifically, the first booster circuit part 63 includes a positive electrode side first booster circuit 63a and the negative electrode side first booster circuit 63b. The positive electrode side first booster circuit 63a of the present embodiment boosts an alternating voltage output from the positive electrode side inverter circuit 62a, and boosts the alternating voltage to, for example, an alternating voltage of 4 times or more and 6 times or less. In the present embodiment, the positive electrode side first booster circuit 63a boosts an alternating voltage of 48 V output from the positive electrode side inverter circuit 62a to an alternating voltage of 250 V.

Meanwhile, the negative electrode side first booster circuit 63b boosts an alternating voltage output from the negative electrode side inverter circuit 62b, and boosts the alternating voltage to, for example, an alternating voltage of 4 times or more and 6 times or less, similar to the positive electrode side first booster circuit 63a. In the present embodiment, the negative electrode side first booster circuit 63b boosts an alternating voltage of −48 V output from the negative electrode side inverter circuit 62b to an alternating voltage of −250 V. It is desirable that the alternating voltage output from the negative electrode side first booster circuit 63b is −60 V or more and −300 V or less. In addition, various well-known circuit configurations can be adopted as specific circuit configurations of the first booster circuit part 63.

In addition, the lithium ion battery 61a of the electric power supply part 60 is connected to an external power source via a connector (not illustrated), and receives the supply of electric power from the external power source, and thus, the lithium ion battery 61a is charged.

The alternating voltage output from the first booster circuit part 63 of the electric power supply part 60 is supplied to the radiation generating part 50 via the cable part 70. The cable part 70 electrically connects the electric power supply part 60 provided within the main body part 20 and the radiation generating part 50 provided at the tip of the arm part 40 to each other, and includes a positive electrode side electric power supply wiring line 70a and a negative electrode side electric power supply wiring line 70b. Each of the positive electrode side electric power supply wiring line 70a and the negative electrode side electric power supply wiring line 70b is formed by covering a conductive member with an insulating member, and extends inside the supporting member 30 and inside the arm part 40. The length of the cable part 70 is, for example, about 3 m and the wiring resistance of the cable part is, for example, about 75 mΩ. Additionally, although not illustrated, the cable part 70 includes a control signal wiring line that supplies a control signal output from the control part 22 to the radiation generating part 50, in addition to the positive electrode side electric power supply wiring line 70a and the negative electrode side electric power supply wiring line 70b.

The radiation generating part 50 is a so-called mono-tank in which a radiation source, a booster circuit, a voltage doubler rectifier circuit, and the like are provided within the housing 51 (refer to FIG. 1). As illustrated in FIG. 4, the radiation generating part 50 of the present embodiment includes an X-ray tube 52 serving as a radiation source, a second booster circuit part 53, and a voltage doubler rectifier circuit part 54.

The second booster circuit part 53 boosts an alternating voltage input via the cable part 70. Specifically, the second booster circuit part 53 includes a positive electrode side second booster circuit 53a, and a negative electrode side second booster circuit 53b. The positive electrode side second booster circuit 53a of the present embodiment boosts the alternating voltage supplied from the positive electrode side electric power supply wiring line 70a, and boosts the alternating voltage to, for example, an alternating voltage of 50 times or more. The positive electrode side second booster circuit 53a of the present embodiment boosts the alternating voltage of 250 V supplied from the positive electrode side electric power supply wiring line 70a, and boosts the alternating voltage to an alternating voltage of 12.5 kV.

Meanwhile, the negative electrode side second booster circuit 53b boosts the alternating voltage supplied from the negative electrode side electric power supply wiring line 70b, and boosts the alternating voltage to, for example, an alternating voltage of 50 times or more, similar to the positive electrode side second booster circuit 53a. The negative electrode side second booster circuit 53b of the present embodiment boosts the alternating voltage of −250 V supplied from the negative electrode side electric power supply wiring line 70b to an alternating voltage of −12.5 kV. In addition, various well-known circuit configurations can be adopted as specific circuit configurations of the second booster circuit part 53.

Additionally, in the present embodiment, as described above, the two booster circuit parts of the first booster circuit part 63 and the second booster circuit part 53 are provided. However, the invention is not necessarily limited to such a configuration. Only one of the booster circuit parts may be provided so that an alternating voltage is boosted.

The voltage doubler rectifier circuit part 54 doubles and rectifies an alternating voltage output from the second booster circuit part 53. Specifically, the voltage doubler rectifier circuit part 54 includes a positive electrode side voltage doubler rectifier circuit 54a and a negative electrode side voltage doubler rectifier circuit 54b. The positive electrode side voltage doubler rectifier circuit 54a doubles and rectifies the alternating voltage output from the positive electrode side second booster circuit 53a, and rectifies the alternating voltage to, for example, a positive direct current voltage of 4 times. The positive electrode side voltage doubler rectifier circuit 54a of the present embodiment rectifies the alternating voltage of 12.5 kV boosted by the positive electrode side second booster circuit 53a to a direct current voltage of 50 kV.

Meanwhile, the negative electrode side voltage doubler rectifier circuit 54b doubles and rectifies the alternating voltage output from the negative electrode side second booster circuit 53b, and rectifies the alternating voltage to, for example, a negative direct current voltage of 4 times. The negative electrode side voltage doubler rectifier circuit 54b of the present embodiment rectifies the alternating voltage of −12.5 kV boosted by the negative electrode side second booster circuit 53b to a direct current voltage of −50 kV. In addition, the specific circuit configuration of the voltage doubler rectifier circuit part 54 is not limited to the circuit configuration illustrated in FIG. 4, and various well-known circuit configurations can be adopted.

The X-ray tube 52 generates radiation by applying a direct current voltage output from the voltage doubler rectifier circuit part 54. In the present embodiment, as described above, the direct current voltage of 50 kV is supplied to a positive electrode side of the X-ray tube 52 by the positive electrode side voltage doubler rectifier circuit 54a, and the direct current voltage of −50 kV is supplied to a negative electrode side of the X-ray tube 52 by the negative electrode side voltage doubler rectifier circuit 54b. As a result, the direct current voltage of 100 kV is applied to the X-ray tube 52.

The exposure switch 90 receives an emission (exposure) instruction of the radiation from the radiation generating part 50. In addition, in the present embodiment, the exposure switch 90 is equivalent to an emission instruction receiving part of the invention. As illustrated in FIG. 4, the exposure switch 90 of the present embodiment includes an exposure SW1 and an exposure SW2. The exposure SW1 receives an emission preparation instruction of radiation, and the exposure SW2 receives an emission instruction of radiation.

In a case where the exposure SW1 is turned on by a user, the switch element 61c is turned on by the battery control part 64, and thus, the capacitor 61b is charged by the lithium ion battery 61a. Additionally, in a case where the exposure SW2 is turned on by the user, the switch element 61c is turned off by the battery control part 64, and thus, a discharge voltage is output from the capacitor 61b.

In addition, in the present embodiment, the two separate switches of the exposure SW1 and the exposure SW2 are provided. However, the configuration of the exposure switch 90 is not limited to this. For example, a switch that receives two push states of half push and full push may be used, a switch element 61c may be turned on in the case of the half push, and the switch element 61c may be turned off in the case of the full push.

Additionally, the exposure switch 90 may be provided in an input part 24 in the monitor 23 to be described below, or may be provided separately from the monitor 23.

Additionally, in the present embodiment, charging to the capacitor 61b by the lithium ion battery 61a and discharging from the capacitor 61b are switched therebetween according to the operation of the exposure SW1 and the exposure SW2 by a user. However, the invention is not limited to this. A control function of switching the connection between the lithium ion battery 61a and the capacitor 61b by determining an imaging menu registered by an engineer may be added. As the imaging menu, for example, there is an imaging menu for performing short-time X-ray imaging multiple times in a short time. In a case where this imaging menu is selected, it is desirable to perform discharging from the capacitor 61b to perform radiation exposure, in a state where the lithium ion battery 61a and the capacitor 61b are connected, that is, in a charging state. In addition, in this case, it is desirable to design the capacity of the capacitor 61b such that a voltage drop on an electric power supply side occurring at the time of the discharging from the capacitor 61b falls within a usable range of the lithium ion battery 61a.

Additionally, the battery control part 64 monitors a terminal voltage of the capacitor 61b. Then, in a case where the switch element 61c is turned on to charge the capacitor 61b and the terminal voltage of the capacitor 61b becomes equal to or more than a preset threshold value, a light-emitting part 91 is caused to emit light. By emitting light from the light-emitting part 91, it is possible to notify a user that the charging of the capacitor 61b is completed. Hence, the user can check light emission of the light-emitting part 91 to turn on the exposure SW2, and can efficiently perform the charging to the capacitor 61b and the exposure of radiation. As the light-emitting part 91, for example, a light emitting diode (LED) can be used. In addition, in the present embodiment, the light-emitting part 91 is caused to emit light by monitoring the terminal voltage of the capacitor 61b. However, the invention is not limited to this. For example, the light-emitting part 91 may be caused to emit light in a case where the time after the exposure SW1 is turned on is measured and the measured time becomes equal to or more than a preset threshold value. Although a threshold value of the measured time also depends on a charging speed to the capacitor 61b and the capacity of the capacitor 61b, it is desirable that the threshold value is 0.8 seconds or more and 4 seconds or less.

Additionally, in the above description, the light-emitting part 91 is turned on in a case where the charging of the capacitor 61b is completed. However, the light-emitting part 91 may be caused to emit light not only in the case where the charging to the capacitor 61b is completed but also in a case where it is detected that, for example, other radiation exposure preparation operations, such as voltage application to a filament, are completed.

Additionally, in the present embodiment, the light-emitting part 91 is equivalent to a notification part of the invention. However, the configuration of the notification part is not limited to this. For example, when the charging of the capacitor 61b is completed, sound may be emitted, or a message may be displayed on the monitor 23.

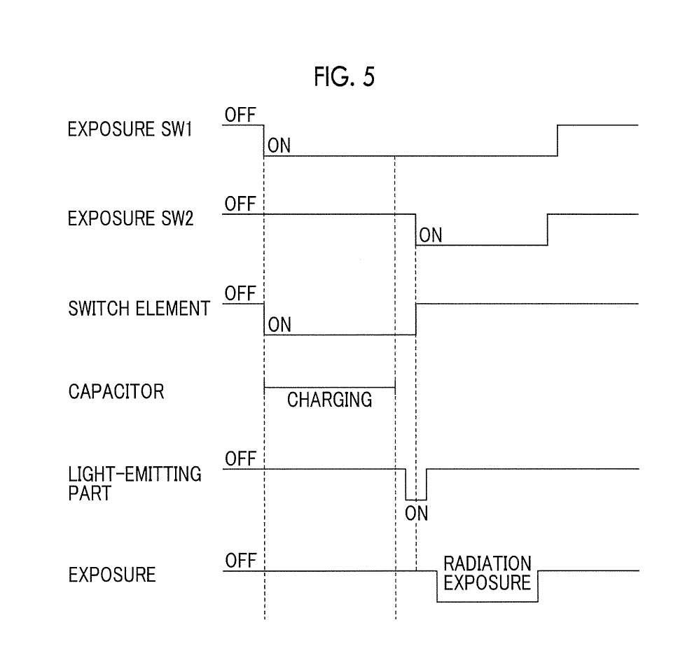

Here, the operation of the radiation irradiation device 1 from the charging to the capacitor 61b of the battery part 61 to the exposure of radiation will be described, referring to a timing chart illustrated in FIG. 5.

First, the exposure SW1 is turned on by a user, and accordingly, the switch element 61c is turned on and the charging of the capacitor 61b is started. In a case where the charging of the capacitor 61b proceeds and the terminal voltage of the capacitor 61b becomes equal to or more than a threshold value of a voltage, the light-emitting part 91 is controlled by the battery control part 64, and the light-emitting part 91 is turned on.

Then, the user turns on the exposure SW2 after ON of the light-emitting part 91 is checked. The switch element 61c is turned off by ON state of the exposure SW2, and thus, the discharge voltage of the capacitor 61b is supplied to the radiation generating part 50 and radiation is radiated from the radiation generating part 50.

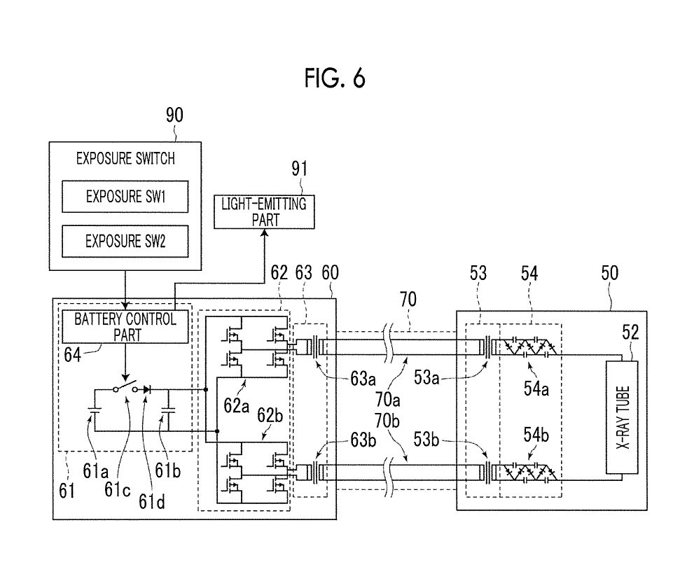

In addition, as for the battery part 61, a diode element 61d may be further provided, as illustrated in FIG. 6, such that the electric charge charged by the capacitor 61b does not flow back to the lithium ion battery 61a side. Although the diode element 61d is equivalent to a reverse current suppressing part of the invention, the reverse current suppressing part is not limited to the diode element 61d, and other well-known elements or circuits can be used.

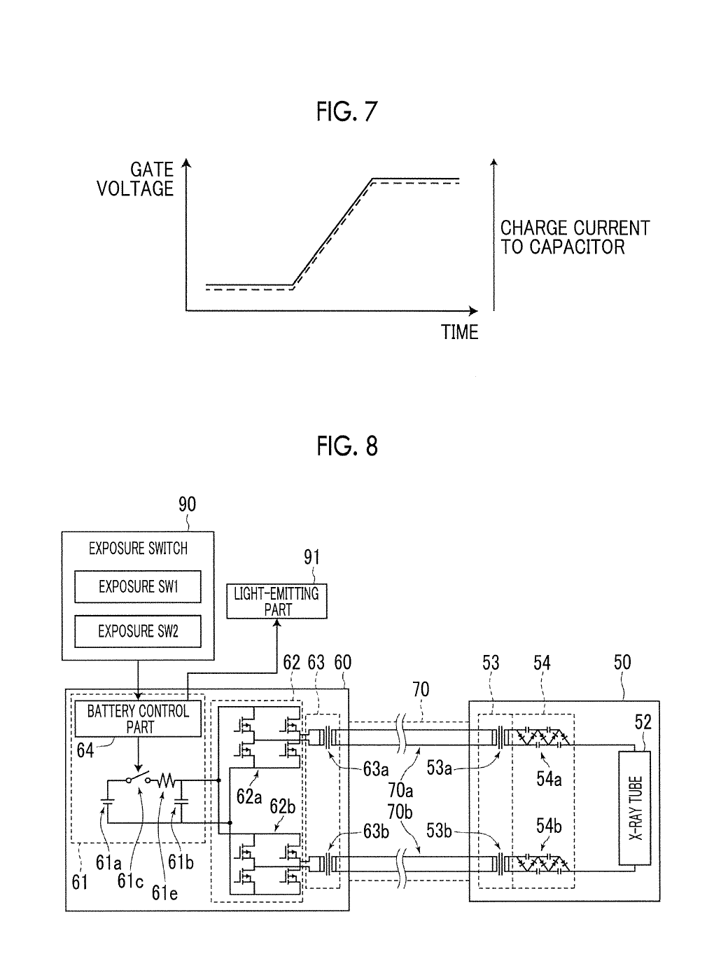

Additionally, in order to reduce an inrush current when charging the capacitor 61b from the lithium ion battery 61a, current limiting may be performed. Specifically, a gate voltage to be applied to the switch element 61c by the battery control part 64 may be gradually increased according to the lapse of time, as illustrated by a solid line of FIG. 7. By controlling the waveform of the gate voltage as illustrated by a solid line of FIG. 7, the waveform of a current flowing into the capacitor 61b can be controlled as illustrated by a dotted line of FIG. 7, and the inrush current to the capacitor 61b can be suppressed.

Additionally, in a case where a relay switch is used as the switch element 61c, in order to reduce the inrush current when the capacitor 61b is charged from the lithium ion battery 61a, a resistance element 61e may be connected to the capacitor 61b in series, as illustrated in FIG. 8.

In addition, although the battery control part 64 and the resistance element 61e that apply the above-described gate voltage are equivalent to an inrush current suppressing part of the invention, the inrush current suppressing part is not limited to this, and other well-known elements or well-known circuits be used.

Returning to FIGS. 1 and 2, an L-shaped radiation source attachment part 32 is provided at a tip (one end) of the arm part 40. The radiation generating part 50 is attached to the one end of the arm part 40 via the radiation source attachment part 32. As illustrated in FIGS. 1 and 2, the cable part 70 taken out from the one end of the arm part 40 is connected to the radiation generating part 50 via a connector.

The radiation generating part 50 is connected to the radiation source attachment part 32 so as to be rotationally movable with an axis AX2 as a rotational movement axis. The rotational movement axis AX2 is an axis that extends in the leftward-rightward direction (x direction). In addition, the radiation source attachment part 32 holds the radiation generating part 50 such that the radiation generating part 50 moves rotationally via a friction mechanism. For this reason, the radiation generating part 50 is rotationally movable by applying a certain degree of strong external force, does not move rotationally unless an external force is applied, and maintains a relative angle with respect to the arm part 40.

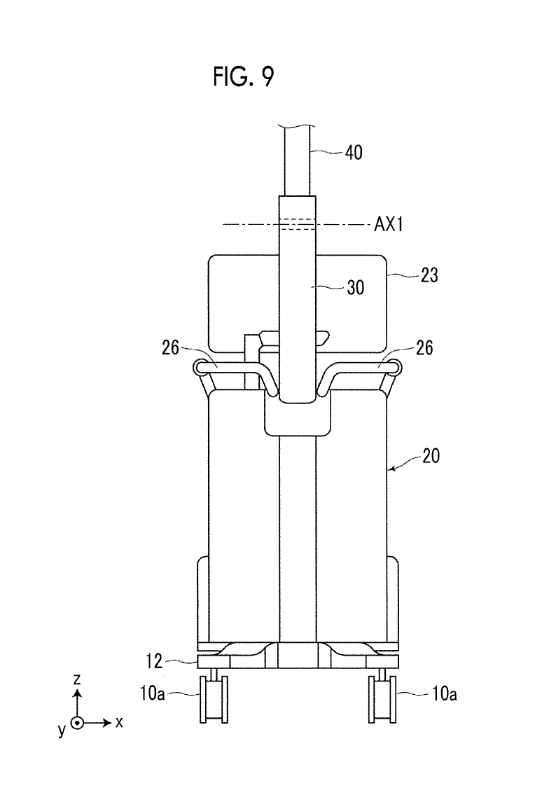

Additionally, the monitor 23 is attached to an upper surface of the housing 21. Additionally, a handle part 26 for pushing or pulling the radiation irradiation device 1 is attached to an upper part of the housing 21. The handle part 26 is provided so as to go around the housing 21, and is configured so as to be capable of being held not only from a rear side of the radiation irradiation device 1 but also from a front side or a lateral side. FIG. 9 is a view of the radiation irradiation device 1 as seen from the front. As illustrated in FIG. 9, the handle part 26 is provided so as to go around to a front side of the main body part 20.

The monitor 23 consists of a liquid crystal panel or the like, and displays a radiation image acquired by imaging of a subject, and various kinds of information required for the control of the radiation irradiation device 1. Additionally, the monitor 23 includes the touch panel type input part 24, and receives input of various instructions required for the operation of the radiation irradiation device 1. Specifically, input for setting of imaging conditions and input for imaging, that is, emission of radiation, can be received. The monitor 23 is attached to the upper surface of the housing 21 so as to be capable of changing the inclination and the rotational position of a display surface with respect to the horizontal direction. Additionally, instead of the touch panel type input part 24, buttons for performing various operations may be included as the input part.

One end of the supporting member 30 is connected to the other end of the arm part 40. The arm part 40 is connected to the supporting member 30 so as to be rotationally movable with an axis AX1 as a rotational movement axis. The rotational movement axis AX1 is an axis that extends in the leftward-rightward direction (x direction). The arm part 40 moves rotationally in a direction of arrow A illustrated in FIG. 2 such that an angle formed with the supporting member 30 is changed about the rotational movement axis AX1.

A rotational movement part 31 having the rotational movement axis AX1 holds the arm part 40 such that the arm part 40 moves rotationally via the friction mechanism. For this reason, the arm part 40 is rotationally movable by applying a certain degree of strong external force, does not move rotationally unless an external force is applied, and maintains a relative angle with respect to the supporting member 30.

In addition, although the rotational movement of the arm part 40 and the radiation generating part 50 is performed via the friction mechanism, rotational movement positions of these parts may be fixed by a well-known locking mechanism. In this case, the rotational movements of the arm part 40 and the radiation generating part 50 become possible by releasing the locking mechanism. The rotational movement positions can be fixed by locking the locking mechanism at desired rotational movement positions.

The other end of the supporting member 30 is connected to the surface of the main body part 20 on the front side. The supporting member 30 is provided so as to be fixed with respect to the main body part 20, and is attached so as to be non-rotatable with respect to the main body part 20. In the present embodiment, as described above, the orientation of the arm part 40 can be freely changed together with the main body part 20 by the revolution of the first casters 10a and the second casters 10b. Thus, it is not necessary to make the supporting member 30 have a degree of freedom, and a simpler configuration can be adopted. However, the invention is not limited to this, and the supporting member 30 may be configured to rotate with emphasis on handleability. That is, the supporting member 30 may be configured so as to be rotatable with an axis passing through the center of the portion of the supporting member 30 connected to the main body part 20 and extending in the vertical direction as a rotation axis.

In the present embodiment, when a subject is imaged, as illustrated in FIG. 2, the imaging is performed by arranging a radiation detector 80 under a subject H that lies on ones' back on a bed 3 and irradiating the subject H with the radiation emitted from the radiation generating part 50. In addition, the radiation detector 80 and the radiation irradiation device 1 are connected together with or without wires. Accordingly, the radiation image of the subject H acquired by the radiation detector 80 is directly input to the radiation irradiation device 1.

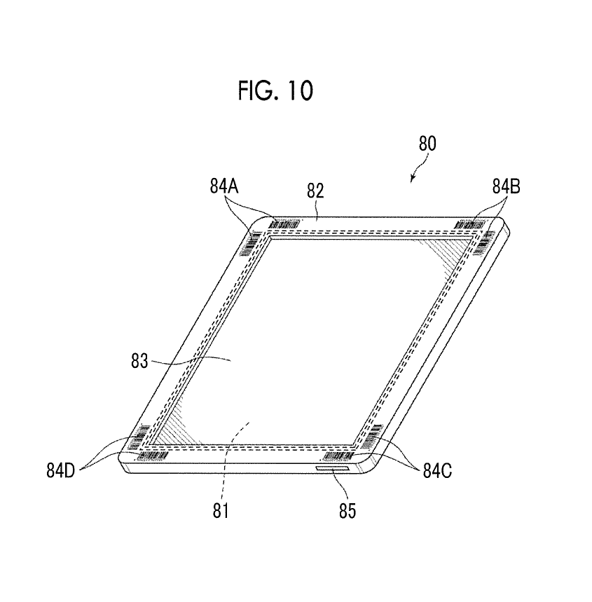

Here, a radiation detector 80 will be briefly described with reference to FIG. 10. FIG. 10 is an external perspective view of the radiation detector as seen from a front surface that is a radiation detection surface side. As illustrated in FIG. 10, the radiation detector 80 is a cassette type radiation detector including a housing 82 that has a rectangular flat plate shape and houses a detecting part 81. The detecting part 81 includes a scintillator (fluorescent body) that converts incident radiation into visible light as is well known, and a thin film transistor (TFT) active matrix substrate. A rectangular imaging region where a plurality of pixels that accumulate electrical charge according to the visible light from the scintillator are arrayed is formed on the TFT active matrix substrate.

Additionally, the housing 82 includes a round-chamfered metallic frame. A gate driver that gives a gate pulse to a gate of a TFT to switch the TFT, an imaging control part including a signal processing circuit that converts an electrical charge accumulated in a pixel into an analog electrical signal representing an X-ray image to output the converted signal, and the like in addition to the detecting part 81 are built in the housing. Additionally, the housing 82 has, for example, a size based on International Organization for Standardization (ISO) 4090:2001 that is substantially the same as a film cassette, an imaging plate (IP) cassette, and a computed radiography (CR) cassette.

A transmission plate 83 that allows radiation to be transmitted therethrough is attached to a front surface of the housing 82. The transmission plate 83 has a size that substantially coincides with a detection region of radiation in the radiation detector 80, and is formed of a carbon material that is lightweight, has high rigidity, and has high radiation transmissivity. In addition, the shape of the detection region is the same oblong shape as the shape of the front surface of the housing 82. Additionally, the portion of the frame of the housing 82 protrudes from the transmission plate 83 in a thickness direction of the radiation detector 80. For this reason, the transmission plate 83 is not easily damaged.

Markers 84A to 84D showing identification information for identifying the radiation detector 80 are applied to four corners of the front surface of the housing 82. In the present embodiment, the markers 84A to 84D consist of two bar codes that are orthogonal to each other, respectively.

Additionally, a connector 85 for charging the radiation detector 80 is attached to a side surface of the housing 82 on the markers 84C, 84D side.

When the radiation irradiation device 1 according to the present embodiment is used, the operator rotationally moves the arm part 40 around the rotational movement axis AX1 in an illustrated counterclockwise direction from an initial position of the arm part 40 illustrated in FIG. 1, and thus, the radiation generating part 50 is moved to a target position immediately above the subject H, as illustrated in FIG. 2. The radiation image of the subject H can be acquired by driving the radiation generating part 50 according to an instruction from the input part 24 to irradiate the subject H with radiation and detecting the radiation transmitted through the subject H, using the radiation detector 80, after the radiation generating part 50 is moved to the target position.

In addition, as the radiation detector 80, as described above, it is desirable to use a radiation detector in which the scintillator and the TFT active matrix substrate including light receiving elements are laminated and which receives irradiation of radiation from a TFT active matrix substrate side (a side opposite to a scintillator side). By using such a high-sensitivity radiation detector 80, a low-output radiation source can be used as the radiation generating part 50, and the weight of the radiation generating part 50 can be made light. In addition, generally, the radiation source output of the radiation generating part 50 and the weight of the radiation generating part 50 are in a proportional relation.

Since the weight of the radiation generating part 50 can be made light as described above, the total weight of the radiation irradiation device 1 can also be made light. Accordingly, by using the revolving casters as the second caster 10b (rear wheels) as in the radiation irradiation device 1 of the present embodiment, the revolution performance of the radiation irradiation device 1 can be improved, and handling can be markedly improved.

In addition, the radiation source output of the radiation generating part 50 is preferably 15 kW or less, and is more preferably 4 kW or less. Additionally, the total weight of radiation irradiation device 1 is preferably 120 kg or less, and is more preferably 90 kg or less.

Next, a configuration in which the radiation detector 80 in the main body part 20 is capable of being housed will be described. As illustrated in FIGS. 1 and 2, the housing 21 of the main body part 20 has a flat surface 21a inclined to a supporting member 30 side, on a surface opposite to a side where the supporting member 30 is attached, and the flat surface 21a is provided with the cradle 25.

An insertion port 25a for inserting the radiation detector 80 is formed in an upper surface of the cradle 25. The insertion port 25a has an elongated shape of a size such that the radiation detector 80 is fitted thereto. In the present embodiment, one end part on a side having the connector 85 of the radiation detector 80 is inserted to the insertion port 25a. Accordingly, this one end part is supported by a bottom part of the cradle 25, and the radiation detector 80 is held by the cradle 25. In this case, a front surface of the radiation detector 80 is directed to a flat surface 21a side.

A connector 25b is attached to the bottom part of the cradle 25. The connector 25b is electrically connected to the connector 85 of the radiation detector 80 when the radiation detector 80 is held by the cradle 25. The connector 25b is electrically connected to the lithium ion battery 61a of the battery part 61. Hence, when the radiation detector 80 is held by the cradle 25, the radiation detector 80 is charged by the lithium ion battery 61a via the connector 85 of the radiation detector 80 and the connector 25b of the cradle 25.

In addition, a configuration in which the radiation detector 80 is chargeable by the lithium ion battery 61a has been described in the present embodiment. As described above, a configuration in which the monitor 23 is chargeable by the lithium ion battery 61a may be adopted. Moreover, a configuration in which an external connector is further provided at the main body part 20 and external instruments other than the monitor are connectable may be adopted. Also, a configuration in which electric power is supplied to an external instrument by the lithium ion battery 61a via the external connector and the external instrument is chargeable may be adopted. As the external instrument, for example, there is a note-type computer used as a console, or the like.

In addition, the radiation irradiation device of the invention does not necessarily include the leg part 10 as in the radiation irradiation device 1 of the above embodiment. Additionally, the configuration of the supporting member 30 and the arm part 40 is not limited to the configuration of the above embodiment, and other configurations may be adopted.

### EXPLANATION OF REFERENCES

1: radiation irradiation device
2: device placement surface
3: bed
10: leg part
10a: first caster
10b: second caster
11: pedestal part
12: foot arm part
12a: tip part
13: pedal part
13a: first pedal
13b: second pedal
20: main body part
21: housing
21a: flat surface
22: control part
23: monitor
24: input part
25: cradle
25a: insertion port
25b: connector
26: handle part
30: supporting member
31: rotational movement part
32: radiation source attachment part
40: arm part
50: radiation generating part
51: housing
52: X-ray tube
53: second booster circuit part
53a: positive electrode side booster circuit
53b: negative electrode side booster circuit
54: voltage doubler rectifier circuit part
54a: positive electrode side voltage doubler rectifier circuit
54b: negative electrode side voltage doubler rectifier circuit
60: electric power supply part
61: battery part
61a: lithium ion battery
61b: capacitor
61c: switch element
61d: diode element
61e: resistance element
62: inverter circuit part
62a: positive electrode side inverter circuit
62b: negative electrode side inverter circuit
63: first booster circuit part
64: battery control part
70: cable part
70a: positive electrode side electric power supply wiring line
70b: negative electrode side electric power supply wiring line
80: radiation detector
81: detecting part
82: housing
83: transmission plate
85: connector
90: exposure switch
91: light-emitting part
AX1: rotational movement axis
AX2: rotational movement axis
H: subject
84A to 84D: marker

What is claimed is:

1. A radiation irradiation device comprising:
a radiation generating part that generates radiation;
a battery part that supplies electric power to the radiation generating part; and
an emission instruction receiving part that receives an emission instruction of the radiation from the radiation generating part,
wherein the battery part has a storage battery, a capacitor connected in parallel to the storage battery, a switching part that performs switching from a state where electric power is supplied from the storage battery to the capacitor to a state where electric power is supplied from the capacitor to the radiation generating part, and
wherein the switching part switches between the states of the electric power supply according to an instruction received at the emission instruction receiving part.

2. The radiation irradiation device according to claim 1,
wherein the switching part has a switch element connected between the storage battery and the capacitor.

3. The radiation irradiation device according to claim 1,
wherein the emission instruction receiving part receives two-step instructions of an emission preparation instruction of the radiation and an emission instruction of the radiation, and
wherein the switching part is brought into a state where electric power is supplied from the storage battery to the capacitor, according to the emission preparation instruction of the radiation.

4. The radiation irradiation device according to claim 1, further comprising:
a notification part that performs notification of charging from the storage battery to the capacitor being completed.

5. The radiation irradiation device according to claim 4,
wherein the notification part includes a light-emitting part that emits light when the charging from the storage battery to the capacitor is completed.

6. The radiation irradiation device according to claim 1, further comprising:
a reverse current suppressing part that suppresses a reverse current from the capacitor to the storage battery.

7. The radiation irradiation device according to claim 6,
wherein the reverse current suppressing part has a diode element.

8. The radiation irradiation device according to claim 1, further comprising:
an inrush current suppressing part that suppresses an inrush current from the storage battery to the capacitor.

9. The radiation irradiation device according to claim 8,
wherein the inrush current suppressing part has a resistance element.

10. The radiation irradiation device according to claim 1,
wherein the capacitor is an electric double layer capacitor.

11. The radiation irradiation device according to claim 1,
wherein the storage battery is a lithium ion battery.

12. The radiation irradiation device according to claim 2,
wherein the emission instruction receiving part receives two-step instructions of an emission preparation instruction of the radiation and an emission instruction of the radiation, and
wherein the switching part is brought into a state where electric power is supplied from the storage battery to the capacitor, according to the emission preparation instruction of the radiation.

13. The radiation irradiation device according to claim 2, further comprising:
a notification part that performs notification of charging from the storage battery to the capacitor being completed.

14. The radiation irradiation device according to claim 2, further comprising:
a reverse current suppressing part that suppresses a reverse current from the capacitor to the storage battery.

15. The radiation irradiation device according to claim 3, further comprising:
a notification part that performs notification of charging from the storage battery to the capacitor being completed.

16. The radiation irradiation device according to claim 3, further comprising:
a reverse current suppressing part that suppresses a reverse current from the capacitor to the storage battery.

17. The radiation irradiation device according to claim 4, further comprising:
a reverse current suppressing part that suppresses a reverse current from the capacitor to the storage battery.

18. The radiation irradiation device according to claim 12, further comprising:
a notification part that performs notification of charging from the storage battery to the capacitor being completed.

19. The radiation irradiation device according to claim 12, further comprising:
a reverse current suppressing part that suppresses a reverse current from the capacitor to the storage battery.

20. The radiation irradiation device according to claim 13,
wherein the notification part includes a light-emitting part that emits light when the charging from the storage battery to the capacitor is completed.
